(19) United States
(12) Patent Application Publication
(10) Pub. No.: US 2025/0324952 A1
(43) Pub. Date: Oct. 23, 2025

(54) METHOD FOR INDUCING DIFFERENTIATION OF UNDIFFERENTIATED GERM CELLS INTO GERM CELL LINEAGE

(71) Applicant: National University Corporation Tokyo University of Marine Science and Technology, Tokyo (JP)

(72) Inventors: Goro YOSHIZAKI, Tokyo (JP); Ryosuke YAZAWA, Tokyo (JP); Aiko SANO, Tokyo (JP)

(73) Assignee: National University Corporation Tokyo University of Marine Science and Technology, Tokyo (JP)

(21) Appl. No.: 18/709,159

(22) PCT Filed: Oct. 27, 2022
(86) PCT No.: PCT/JP2022/040074

§ 371 Date: Nov. 25, 2024

(30) Foreign Application Priority Data
Nov. 11, 2021 (JP) 2021-184335

(51) Int. Cl. A01K 67/02; C12N 5/0735; C12N 5/075; C12N 5/076; C12N 5/10

(52) U.S. Cl. CPC A01K 67/02; C12N 5/0609; C12N 5/061; C12N 5/0611; C12N 5/10; A01K 2227/40; A01K 2267/025; C12N 2506/04; C12N 2510/00

(57) ABSTRACT

Provided is a method for obtaining the germ cell lineage of an oviparous vertebrate more efficiently than conventional techniques. A host oviparous vertebrate is prepared at a developmental stage after the development of black pigmentary cells in the retina and before the formation of multiple layers of germ cells in the genitals, and isolated undifferentiated germ cells from a donor oviparous vertebrate are transplanted into the host oviparous vertebrate.

### BACKGROUND OF THE INVENTION

### Field of the Invention

The present disclosure relates to a method for inducing differentiation of undifferentiated germ cells of an oviparous vertebrate into germ cell lineage.

### Background Art

Conventionally, in vertebrates, genetic modification and cloning of individual animals has been attempted to produce transgenic or cloned animals. However, in oviparous vertebrates, especially in fish, there has been little technology available for transplanting such genetically modified cells or cells intended for cloning into a host individual and inducing their differentiation to obtain genetically modified individuals, compared to other vertebrates.

Under these circumstances, in order to genetically modify or clone individuals of oviparous vertebrates such as fish, and then to breed them or produce cloned animals, techniques have been sought for obtaining genetically modified individuals or cloned animals by transplanting genetically modified or isolated cells into a host individual and then inducing differentiation of these cells to generate a new individual.

As one such technique, a method for inducing differentiation of isolated primordial germ cells from an oviparous vertebrate into a germ cell lineage by transplanting the isolated primordial germ cells from an oviparous vertebrate into an early embryo of another oviparous vertebrate host, and a method for producing a new individual using the resulting germ cell lineage are know (for example, Patent Document 1).

### PRIOR ART LITERATURE

### Patent Literature

[Patent Document 1] JP 2003-235558 A

### SUMMARY OF THE INVENTION

### Problem to be Solved by the Invention

However, coupled with the expected future increase in demand for oviparous vertebrates for research purposes, for securing food resources, and for other reasons, there is a need for a more efficient method for obtaining germ cell lineage of an oviparous vertebrate than conventional techniques.

### Means for Solving the Problem

The present inventors have conducted intensive research on a method for obtaining germ cell lineage of an oviparous vertebrate and have found that germ cell lineage of an oviparous vertebrate can be obtained more efficiently than with conventional techniques, by transplanting isolated undifferentiated germ cells from a donor oviparous vertebrate into an early embryo of a host oviparous vertebrate at a specific range of time point. The present disclosure is based on such findings. That is, the summary of the present disclosure is as follows.

[1] A method for inducing differentiation of isolated undifferentiated germ cells into germ cell lineage, comprising the step of:
(a) preparing a host oviparous vertebrate at a developmental stage after the development of black pigmentary cells in the retina and before the formation of multiple layers of germ cells in the genitals; and
(b) transplanting isolated undifferentiated germ cells from a donor oviparous vertebrate into the host oviparous vertebrate.
   [2] The method according to [1], wherein the isolated undifferentiated germ cells comprise at least one type of cells selected from the group consisting of primordial germ cells, oogonia and spermatogonia.
   [3] The method according to [1] or [2], wherein the transplantation is carried out by transplanting the isolated undifferentiated germ cells into the abdominal cavity of the host oviparous vertebrate.
   [4] The method according to any one of [1] to [3], wherein the isolated undifferentiated germ cells are undifferentiated germ cells obtained by visualizing and isolating undifferentiated germ cells of the donor oviparous vertebrate.
   [5] The method according to [4], wherein the visualization of the undifferentiated germ cells is carried out by incorporating a plasmid in which a marker gene encoding a protein capable of visualizing the undifferentiated germ cells in a living organism is incorporated into the regulatory region of a gene specifically expressed in germ cells of the donor oviparous vertebrate, into the cytoplasm of a fertilized ovum of the donor oviparous vertebrate.
   [6] The method according to [5], wherein the gene specifically expressed in germ cells of the donor oviparous vertebrate is a vasa gene, and the marker gene encoding a protein capable of visualizing the undifferentiated germ cells in a living organism is a gene encoding a fluorescent protein FP.
   [7] The method according to [4], wherein the visualization of the undifferentiated germ cells is carried out by injecting a labeled antibody specifically recognizing an antigen on the cell membrane surface of the germ cells of the donor oviparous vertebrate into the donor oviparous vertebrate and allowing the antigen on the cell membrane surface of the germ cells of the donor oviparous vertebrate to bind to the labeled antibody.
   [8] The method according to any one of [1] to [7], wherein the induction of differentiation of the isolated undifferentiated germ cells into the germ cell lineage is induction of differentiation of primordial germ cells into oocytes or spermatogonia, induction of differentiation of primordial germ cells into ovum or sperm, induction of differentiation of oocytes into ovum or sperm, or induction of differentiation of spermatogonia into ovum or sperm.
   [9] The method according to any one of [1] to [8], wherein the donor oviparous vertebrate and the host oviparous vertebrate are of different strains or species.
   [10] The method according to any one of [1] to [9], wherein the isolated undifferentiated germ cells are genetically modified.
   [11] The method according to any one of [1] to [10], wherein the donor oviparous vertebrate and the host oviparous vertebrate are fish.
   [12] The method according to any one of [1] to [11], wherein the donor oviparous vertebrate is in an embryonic state before or after hatching.
   [13] A method for producing an oviparous vertebrate, comprising the steps of:
(a) inducing differentiation of isolated undifferentiated germ cells from an oviparous vertebrate by the method according to any one of [1] to [12] obtain at least one of ovum and sperm; and
(b) obtaining an individual of an oviparous vertebrate using at least one of the ovum and sperm obtained in the step (a).
   [14] A method for producing a genetically modified oviparous vertebrate, comprising the steps of:
(a) inducing differentiation of isolated undifferentiated germ cells from an oviparous vertebrate having a genetic modification by the method according to any one of [1] to [12] obtain at least one of ovum and sperm having a genetic modification; and
(b) obtaining an individual of an oviparous vertebrate having the genetic modification using at least one of the ovum and sperm having the genetic modification obtained in the step (a).

### EFFECT OF THE INVENTION

According to the present disclosure, by transplanting isolated undifferentiated germ cells into a host oviparous vertebrate at a developmental stage after the development of black pigmentary cells in the retina and before the formation of multiple layers of germ cells in the genitals, the germ cell lineage of an oviparous vertebrate can be obtained more efficiently than with conventional techniques.

### BRIEF DESCRIPTION OF THE DRAWINGS

FIG. 1 shows stereomicroscopic photographs of chub mackerel at each growth stage. The left side of the figure shows photographs of the side of the chub mackerel at each growth stage. The right side of the figure shows photographs of a tissue slice from the abdominal cavity of the chub mackerel at each growth stage. In the figure, the black arrows indicate black pigmentary cells, and the white arrowheads indicate primordial germ cells of the host chub mackerel.

FIG. 2 shows stereomicroscopic photographs of the oriental bonito at each growth stage. The left side of the figure shows photographs of the side of oriental bonito at each growth stage. The right side of the figure shows photographs of a tissue slice from the abdominal cavity of the oriental bonito at each growth stage. In the figure, the black arrows indicate black pigmentary cells, and the white arrowheads indicate primordial germ cells of the host oriental bonito.

FIG. 3 shows stereomicroscopic photographs of ninespine stickleback at each growth stage. The left side of the figure shows photographs of the side of the ninespine stickleback at each growth stage. The right side of the figure shows photographs of a tissue slice from the abdominal cavity of the ninespine stickleback at each growth stage. In the figure, the black arrows indicate black pigmentary cells, and the white arrowheads indicate primordial germ cells of the host ninespine stickleback.

### DETAILED DESCRIPTION OF THE PREFERRED EMBODIMENTS

### Method for Inducing Differentiation of Isolated Undifferentiated Germ Cells Into Germ Cell Lineage

One aspect of the present disclosure provides a method for inducing differentiation of isolated undifferentiated germ cells into germ cell lineage (hereinafter also referred to as the “method of the present disclosure”). Specifically, the method of the present disclosure includes a step of preparing a host oviparous vertebrate at a developmental stage after the development of black pigmentary cells in the retina and before the formation of multiple layers of germ cells in the genitals (step (a)), and a step of transplanting isolated undifferentiated germ cells from a donor oviparous vertebrate into the host oviparous vertebrate (step (b)). Steps (a) and (b) will be described in detail below.

### Step (a)

In step (a), a host oviparous vertebrate is prepared. The host oviparous vertebrate is selected from those in a developmental stage after black pigmentary cells have developed in the retina and before multiple layers of germ cells are formed in the genitals. By transplanting isolated undifferentiated germ cells into a host oviparous vertebrate in such a specific developmental stage (hereinafter also referred to as the “optimum transplantation period”), the success rate of differentiation induction of the transplanted isolated undifferentiated germ cells in the host oviparous vertebrate (survival rate of the isolated undifferentiated germ cells after differentiation induction) can be improved compared to the conventional technology. The reason for this is unclear, but can be inferred as follows. The period during which the transplanted germ cells can be incorporated into the genitals of the host varies depending on the species, but the period limited by the technology of the present disclosure is common across species, ensuring a very short period during which the transplanted germ cells can be engrafted. In addition, in order for the transplanted germ cells to maintain, grow and differentiate, they need to be engrafted quickly into the genitals or the genital gland primordia of the host after transplantation. Therefore, it is considered that the germ cells need to be transplanted at a very limited time.

The term “oviparous vertebrate” refers to a vertebrate in which a fertilized ovum develops outside the mother's body. The type of oviparous vertebrate is not particularly limited as long as the effect of the present disclosure is achieved, and includes, for example, an animal belonging to fish, amphibian, reptile or bird. In one preferred embodiment, an animal belonging to fish is used as the oviparous vertebrate.

The term “host oviparous vertebrate” refers to an oviparous vertebrate into which isolated undifferentiated germ cells are transplanted and which becomes a host and induces the differentiation of the transplanted isolated undifferentiated germ cells into germ cell lineage. The developmental stage of the host oviparous vertebrate is not particularly limited as long as it is in the optimum transplantation period described above, but preferably, an oviparous vertebrate in an embryonic state before or after hatching, more preferably, an oviparous vertebrate in an embryonic state after hatching is used. In an oviparous vertebrate in an embryonic state before or after hatching, immune competence is not established, and immune rejection of the transplanted isolated undifferentiated germ cells is unlikely to occur. Therefore, by using an oviparous vertebrate in an embryonic state before or after hatching as a host, transplantation can be performed without using immunosuppressants, etc., not only when transplanting isolated undifferentiated germ cells from a donor oviparous vertebrate of the same species or strains, but also when transplanting isolated undifferentiated germ cells from a donor oviparous vertebrate of a different species or strains.

The start of the optimum transplantation period is the time when black pigmentary cells (also called “melanophores” or “black pigmentary phores”) develop in the retina. Black pigmentary cells begin to develop in the early stages of growth in an oviparous vertebrate; for example, in fish, they begin to develop at the stage known as larvae immediately after hatching. The presence or absence of development of black pigmentary cells in the retina of an oviparous vertebrate can be confirmed by observation using a stereomicroscope. Specifically, about 50 larvae are observed in a petri dish using a stereomicroscope at 10-20× magnification, and if the retinas of all individuals appear black, it is determined that black pigmentary cells have developed in the retina.

The end of the optimum transplantation period is the time when multiple layers of germ cells are formed in the genitals. In an oviparous vertebrate, as they progress through the stages of growth, germ cells migrate to and are engrafted to the genital ridges that form the genital tract in adults. The germ cells then begin to be covered with the genitals. As the formation of the genitals progresses, the germ cells form layers. Therefore, the time point when multiple layers of germ cells are formed in the genital tract refers to the time point when the formation of germ cells progresses on the surface of the genitals and a layer of germ cells begins to form. The state of germ cells in the genitals of an oviparous vertebrate can be confirmed by observation using a biological microscope.

Thus, in the present disclosure, by transplanting undifferentiated germ cells at the optimum transplantation period as described above, germ cell lineage can be efficiently obtained. In other words, the above-mentioned generation of black pigmentary cells in the retina and the formation of multiple layers of germ cells in the genitals can also be indicators for determining the optimum transplantation period when undifferentiated germ cells are transplanted into an oviparous vertebrate to efficiently induce differentiation into germ cell lineage.

### Step (b)

In step (b), the isolated undifferentiated germ cells from the donor oviparous vertebrate are transplanted into the host oviparous vertebrate at the specific developmental stage prepared in step (a) above. The isolated undifferentiated cells transplanted into the host oviparous vertebrate are induced to differentiate into germ cell lineage in the body of the host oviparous vertebrate.

The donor oviparous vertebrate refers to an oviparous vertebrate that provides (donates) isolated undifferentiated germ cells to a host oviparous vertebrate. The developmental stage of the donor oviparous vertebrate is not particularly limited as long as it has isolated undifferentiated germ cells, but an oviparous vertebrate in an embryonic state before or after hatching is preferably used.

The donor oviparous vertebrate and the host oviparous vertebrate may be of the same strain or different strains. Further, the donor oviparous vertebrate and the host oviparous vertebrate may be of the same species or different species.

The isolated undifferentiated germ cells refer to undifferentiated germ cells isolated and purified from an individual of an oviparous vertebrate. The undifferentiated germ cells are not particularly limited as long as the effects of the present disclosure are achieved, and examples thereof include primordial germ cells, oogonia, spermatogonia, etc. Therefore, in the present disclosure, the isolated undifferentiated germ cells comprise at least one of primordial germ cells, oogonia, and spermatogonia isolated from a host oviparous vertebrate.

Undifferentiated germ cells are a cell population that appear in large numbers during a limited stage in the early development of an oviparous vertebrate, specifically during a limited period before or after hatching. Because undifferentiated germ cells originate from extremely young individuals (embryos) during the early developmental stage of an oviparous vertebrate, they have an inherently extremely high proliferation capacity, and it has been confirmed that they also proliferate extremely quickly within the body of a host oviparous vertebrate after transplantation.

The isolated undifferentiated germ cells used in the present disclosure preferably do not comprise any cells other than undifferentiated germ cells. Therefore, when isolating undifferentiated germ cells from an oviparous, it is preferable to avoid contamination with cells other than undifferentiated germ cells. In addition, when a cell population of undifferentiated germ cells isolated from an oviparous vertebrate comprises cells other than undifferentiated germ cells, it is preferable to remove the cells other than undifferentiated germ cells from the cell population to purify.

In order to avoid the cell population of undifferentiated germ cells isolated from an oviparous vertebrate from being contaminated by cells other than undifferentiated germ cells, and to remove the cells other than undifferentiated germ cells from the cell population to purify, the undifferentiated germ cells are preferably visualized and then isolated and purified from the oviparous vertebrate.

Methods for visualizing undifferentiated germ cells are not particularly limited, and include, for example, the method reported by the present inventors (Int. J. Dev. Biol., 44:323-326, 2000). Specifically, a gene specifically expressed in germ cells of an oviparous vertebrate (Mol. Reprod. Develop., 55:364-371, 2000) is identified, and its regulatory region is used. For example, when the oviparous vertebrate is an animal belonging to fish, examples of genes specifically expressed in germ cells include the vasa gene and the dead end gene. The vasa gene is a gene that has been identified as a causative gene for a mutation in Drosophila that causes infertility in the F1 generation (i.e., no F2 generation is obtained), and is said to have an RNA helicase involved in the translation of mRNA in germ cells, is specifically expressed in germ cells also in fish. Therefore, when the oviparous vertebrate is an animal belonging to fish, the expression of this vasa gene can be used to visualize undifferentiated germ cells. Another method for visualizing undifferentiated germ cells includes a method for visualizing a germ cell population comprising undifferentiated germ cells using an antibody that specifically recognizes an antigen on the surface of the cell membrane of germ cells of a donor oviparous vertebrate. In this method, an antibody that specifically recognizes an antigen on the surface of the cell membrane of germ cells of a donor oviparous vertebrate is labeled, and the resulting labeled antibody is inoculated into a donor oviparous vertebrate, thereby binding the antigen on the surface of the cell membrane of germ cells of the donor oviparous vertebrate to the labeled antibody, thereby visualizing undifferentiated germ cells. In this method, examples of the antibody label include fluorescent proteins. Examples of the antibodies used in this method include the antibodies disclosed in the patent application of the present inventors (Japanese Patent Publication No. 2018-203662). These methods for visualizing undifferentiated germ cells can be used whether the host oviparous vertebrate and the donor oviparous vertebrate are of the same species or different species, but when the host oviparous vertebrate and the donor oviparous vertebrate are of the same species, the former method is preferably used. On the other hand, when the host oviparous vertebrate and the donor oviparous vertebrate are of different species, the latter method is preferably used.

In the visualization of undifferentiated germ cells, in order to label and isolate undifferentiated germ cells in the living body of a donor oviparous vertebrate by expression of a gene specifically expressed in undifferentiated germ cells, a plasmid in which a marker gene encoding, for example, a fluorescent protein capable of visualizing undifferentiated germ cells in the living body is incorporated into the regulatory region of a gene specifically expressed in the germ cells of an oviparous vertebrate, such as the vasa gene, is introduced into the cytoplasm of a fertilized ovum of an oviparous vertebrate. Examples of such a marker gene include genes encoding fluorescent proteins, specifically, genes encoding green fluorescent protein (GFP) derived from Aequorea victoria and genes encoding enhanced green fluorescent protein (EGFP).

The undifferentiated germ cells visualized as described above can be isolated from the genitals of the donor oviparous vertebrate by known methods. For example, when the undifferentiated germ cells to be isolated are primordial germ cells, a genital ridge comprising the undifferentiated germ cells is extracted from a hatched embryo, treated with a protease to dissociate the cells, and the primordial germ cells expressing the fluorescent protein as described above can be isolated and obtained from the dissociated cell population using a cell sorter or the like.

When the undifferentiated germ cells to be isolated are oogonia, they can be extracted from ovarian tissue comprising oogonia by treatment with proteolytic enzymes, and oogonia expressing a fluorescent protein can be obtained by the same method as in the case of primordial germ cells described above.

When the undifferentiated germ cells to be isolated are spermatogonia, they can be extracted from testicular tissue comprising spermatogonia by treatment with proteolytic enzymes, and spermatogonia expressing a fluorescent protein can be obtained by the same method as in the case of primordial germ cells described above.

The undifferentiated germ cells isolated as described above may be used as they are, or may be genetically modified before use. As a method for genetic modification, a known method for introducing genes or a known method for modifying genes may be used. Specific examples of the method for genetic modification include the microinjection method, the electroporation method, the gene gun method (the particle gun method), and the like.

When undifferentiated germ cells that have not been genetically modified are used, the resulting germ cell lineage is considered to have the same genetic constitution as the donor oviparous vertebrate. Therefore, germ cell lineage having the same genetic constitution as the donor oviparous vertebrate of undifferentiated germ cells can be efficiently obtained by using undifferentiated germ cells that have not been genetically modified. Then, an individual having the same genetic constitution as the donor oviparous vertebrate can be efficiently produced by breeding using such germ cell lineage having the same genetic constitution as the donor oviparous vertebrate.

On the other hand, when undifferentiated germ cells that have been genetically modified are used, the resulting germ cell lineage is considered to have the genetic modification. Therefore, germ cell lineage having the genetic modification can be efficiently obtained by using undifferentiated germ cells with a desired genetic modification. Then, an individual having a desired genetic modification can be efficiently produced by breeding using such germ cell lineage having the genetic modification.

The transplantation of isolated undifferentiated germ cells derived from a donor oviparous vertebrate refers to transplanting isolated undifferentiated germ cells from a donor oviparous vertebrate into the body of a host oviparous vertebrate. The site in the body of the host oviparous vertebrate into which isolated undifferentiated germ cells from a donor oviparous vertebrate are transplanted is not particularly limited as long as the effects of the present disclosure are achieved, and examples thereof include the ovary, testis, intraperitoneal cavity, and the like. When undifferentiated germ cells are transplanted into the abdominal cavity, the site is not particularly limited, and for example, they are transplanted behind the mesentery in the abdominal cavity. The undifferentiated germ cells transplanted into the ovary or testis are induced to differentiate into germ cell lineage in the ovary or testis as they are. On the other hand, the undifferentiated germ cells transplanted into the abdominal cavity are attracted by a germ cell attractant released from the ovary or testis to move to the ovary or testis, and are induced to differentiate into germ cell lineage in the ovary or testis. The method of transplantation is not particularly limited as long as it is a method commonly used in cell transplantation, and the transplantation can be performed by, for example, microinjection, etc.

Induction of differentiation into the germ cell lineage refers to that the degree of differentiation as a germ cell advances, and specific examples include induction of differentiation of primordial germ cells into oocytes or spermatogonia, induction of differentiation of primordial germ cells into ovum or sperm, induction of differentiation of oocytes into ovum or sperm, or induction of differentiation of spermatogonia into ovum or sperm.

Thus, according to the method of the present disclosure, it is possible to obtain germ cell lineage having the same genetic constitution as that of conventional oviparous vertebrates, and germ cell lineage having a genetic constitution different from that of conventional oviparous vertebrates more efficiently than with conventional techniques.

One embodiment of the method of the present disclosure includes, for example, use of an oviparous vertebrate as a surrogate. Specifically, by transplanting undifferentiated germ cells isolated from a donor oviparous vertebrate into a host oviparous vertebrate of a different strain or species, it is possible to induce differentiation of ovum and sperm derived from a different strain or species of oviparous vertebrate (i.e., the donor oviparous vertebrate) in the body of the host oviparous vertebrate. For example, when the donor oviparous vertebrate is a large animal and the host oviparous vertebrate is a small closely related species, by transplanting undifferentiated germ cells of the large donor oviparous vertebrate into the small host oviparous vertebrate, it is possible to induce differentiation into the germ cell lineage of the large donor oviparous vertebrate in the body of the small host oviparous vertebrate. In general, since a small oviparous vertebrate are easier to grow and require less cost than a large oviparous vertebrate, in the case, the cost of obtaining the germ cell lineage of the large oviparous vertebrate can be reduced.

Another embodiment of the method of the present disclosure includes use for the preservation of genetic resources of oviparous vertebrates. Specifically, the germ cell lineage of an animal species classified as a rare or endangered species is preserved, and if necessary, the germ cell lineage is transplanted into a related species that is easy to grow as a host oviparous vertebrate, thereby obtaining ovum and sperm of the animal species classified as a rare or endangered species. Furthermore, the obtained ovum and sperm can be used to obtain individuals of the animal species classified as a rare or endangered species. Furthermore, if various useful varieties and strains are produced in the future through technologies such as gene introduction, the germ cell lineage can be preserved as described above, ovum and sperm can be obtained as necessary, and then individuals can be obtained using them, thereby making it possible to maintain useful varieties and strains without the need for successive breeding of individuals.

### Method for Producing Oviparous Vertebrates

One aspect of the present disclosure provides a method for producing an oviparous vertebrate, which comprises a step of inducing differentiation of isolated undifferentiated germ cells from an oviparous vertebrate by the above-mentioned production method of the present disclosure to obtain at least one of ovum and sperm (Step (a)), and a step of obtaining an individual of an oviparous vertebrate using at least one of the ovum and sperm obtained in step (a) (Step (b)). Steps (a) and (b) will be described in detail below.

### Step (a)

In step (a), differentiation of isolated undifferentiated germ cells from an oviparous vertebrate is induced by the above-mentioned production method of the present disclosure to obtain at least one of ovum and sperm. The obtained ovum and sperm may be obtained by transplanting the isolated undifferentiated germ cells from a donor oviparous vertebrate into a host oviparous vertebrate without genetic modification, or may be obtained by transplanting the isolated undifferentiated germ cells from a donor oviparous vertebrate into a host oviparous vertebrate after genetic modification.

When isolated undifferentiated germ cells that have not been genetically modified are used, the resulting ovum and sperm have the same genetic constitution as the host oviparous vertebrate. On the other hand, when isolated undifferentiated germ cells that have been genetically modified are used, the resulting ovum and sperm have a genetic constitution that includes the genetic modification.

### Step (b)

In step (b), the ovum and/or sperm obtained in the above-mentioned step (a) are used to obtain an individual oviparous vertebrate. Specifically, the obtained ovum and/or sperm are fertilized to obtain fertilized ovum, and the fertilized ovum are developed to obtain an individual oviparous vertebrate. The method for fertilizing the ovum and/or sperm to obtain fertilized ovum and the method of developing the fertilized ovum are not particularly limited, and known methods can be used.

When the ovum and sperm used in this step are obtained from the same host oviparous vertebrate, the oviparous vertebrate obtained in this step will have the same genetic constitution as the host oviparous vertebrate. On the other hand, when the ovum and sperm used in this step are obtained from different host oviparous vertebrates, the oviparous vertebrate obtained in this step will have a genetic constitution different from that of the host oviparous vertebrate. In addition, when the ovum or sperm obtained in step (a) are fertilized with the sperm or ovum of a natural oviparous vertebrate (i.e., an oviparous vertebrate not obtained by the method of the present disclosure), the resulting oviparous vertebrate will have a genetic constitution different from that of the host oviparous vertebrate.

Thus, according to the production method disclosed herein, it is possible to more efficiently obtain an oviparous vertebrate having the same genetic constitution as conventionally existing oviparous vertebrates, and an oviparous vertebrate having a genetic constitution different from that of conventionally existing oviparous vertebrates, as compared to conventional techniques.

One embodiment of the production method of the present disclosure is, for example, use in breeding (breed improvement) of oviparous vertebrates. Specifically, an individual having a genetic modification of interest by genetically modifying in vitro isolated undifferentiated germ cells, then transplanting them into the body of a host oviparous vertebrate, inducing differentiation to obtain ovum and sperm, and then fertilizing them.

Another embodiment of the production method of the present disclosure is use in for function analysis of oviparous vertebrates. Specifically, by performing gene targeting on undifferentiated germ cells of an oviparous vertebrate and then obtaining an individual oviparous vertebrate using the undifferentiated germ cells, it is possible to produce knockout animals in which the function of the gene targeted by gene targeting has been destroyed, or knockin animals in which the sequence of the gene has been modified. This makes it possible to clarify the role of genes whose functions are unknown.

### EXAMPLES

The present disclosure will be specifically described below based on examples, but the present disclosure is not limited to these examples.

### Example 1: Differentiation Induction of Testicular Germ Cells in Chub Mackerel

### (Isolation of Testicular Germ Cells From Chub Mackerel)

To isolate testicular germ cells from chub mackerel, juvenile chub mackerel with testes having undifferentiated germ cells were prepared.

Next, the testes extracted from the chub mackerel were treated with proteolytic enzymes to dissociate the cells, thereby obtaining a cell population comprising germ cells.

### (Induction of Differentiation of Isolated Testicular Cells Into Germ Cell Lineage)

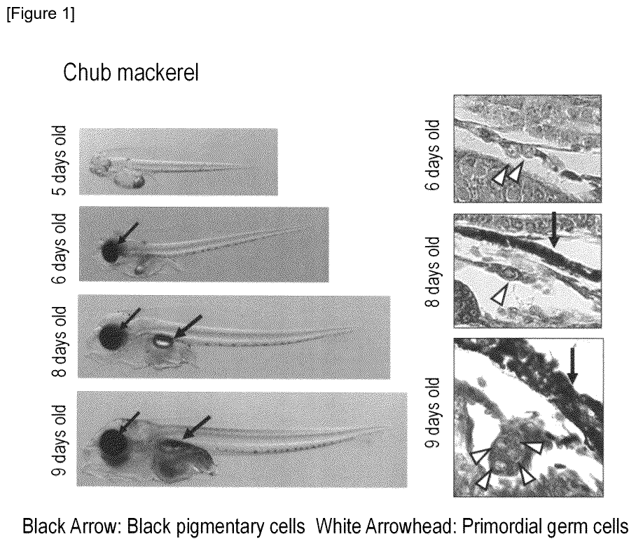

The isolated testicular cells were transplanted into chub mackerel larvae at each growth stage at 5 days, 6 days, 8 days and 9 days after fertilization (5 days old, 6 days old, 8 days old and 9 days old, respectively). Specifically, about 10 isolated primordial germ cells were extracted using a glass micropipette equipped to a microinjector, and transplanted by injecting them into the abdominal cavity of each chub mackerel larva behind the mesentery. In chub mackerel larvae at 5 days after fertilization, black pigmentary cells were not observed in the retina, and multiple layers of germ cells were neither observed in the genitals. In chub mackerel larvae at 6 and 8 days after fertilization, black pigmentary cells were observed in the retina, and multiple layers of germ cells were not observed in the genital organs. In chub mackerel larvae at 9 days after fertilization, black pigmentary cells were observed in the retina, and multiple layers of germ cells were also observed in the genitals. The presence or absence of black pigmentary cells in the retina was observed by observing 50 chub mackerel larvae in a petri dish under a stereomicroscope at 10-20× magnification, and if the retinas of all individuals appeared black, it was determined that black pigmentary cells had developed in the retina. Stereomicroscopic photographs of tissue slices from the side and abdominal cavity of chub mackerel at each growth stage are shown in FIG. 1. The abdominal cavity tissue slices were fixed in Bouin's solution, and the cells were stained with hematoxylin-eosin staining.

After transplantation, it was confirmed by microscopic or histological observation of the genital gland that the transplanted germ cells from testis were induced to differentiate into germ cell lineage. In addition, in chub mackerel larvae into which testicular cells were transplanted at 5th day after fertilization, the transplantation efficiency of the differentiated germ cell lineage was almost 0%. In addition, in chub mackerel larvae into which primordial germ cells were transplanted at 6th and 8th days after fertilization, the transplantation efficiency of the differentiated germ cell lineage was 4.8±1.1% and 11.3±8.7%, respectively. In addition, in chub mackerel larvae into which primordial germ cells were transplanted at 9th day after fertilization, the transplantation efficiency of the differentiated germ cell lineage was 1.4±1.4%. The “transplantation efficiency” is a value obtained by multiplying the engraftment rate by the survival rate, where the percentage of engrafted undifferentiated germ cells among the total number of transplanted undifferentiated germ cells is the “engraftment rate,” and the percentage of germ cell lineages that were induced to differentiate and survive among the total number of engrafted undifferentiated germ cells is the “survival rate.” The above values of each transplantation efficiency are the average values of the transplantation efficiency values of three tests (n=3). These results showed that germ cell lineage can be efficiently obtained by transplanting isolated testicular cells into chub mackerel at a developmental stage after black pigmentary cells have developed in the retina and before multiple layers of germ cells are formed in the genitals.

### Example 2: Differentiation Induction of Primordial Germ Cells in Oriental Bonito

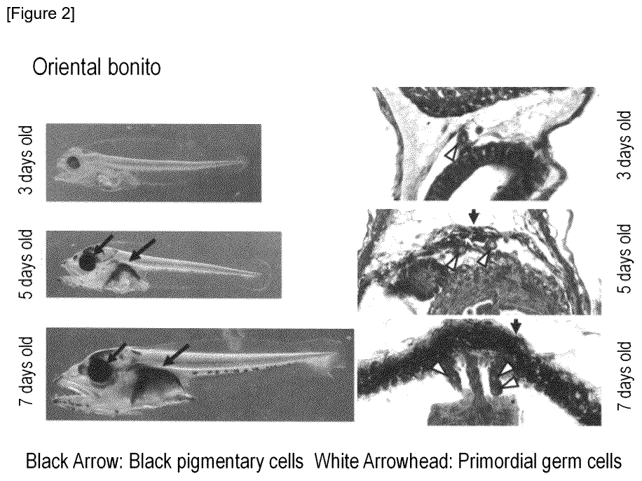

Following the same procedure as in Example 1, germ cells from testis of oriental bonito were isolated and transplanted into oriental bonito larvae at each growth stage after fertilization (3 days old, 5 days old and 7 days old, respectively). In oriental bonito larvae at 3 days after fertilization, black pigmentary cells were not observed in the retina, and multiple layers of germ cells were neither observed in the genitals. On the other hand, in oriental bonito larvae at 5 days and 7 days after fertilization, black pigmentary cells were observed in the retina, and multiple layers of germ cells were not observed in the genitals. Photographs of oriental bonito at each growth stage are shown in FIG. 2.

After transplantation, it was confirmed by microscopic or histological observation of the genital gland that the transplanted germ cells from testis were induced to differentiate into germ cell lineage. In addition, in oriental bonito larvae into which primordial germ cells were transplanted at 5th day after fertilization, the transplantation efficiency of the differentiated germ cell lineage was 33.3%, which was higher than that of primordial germ cells transplanted at 3rd day after fertilization. In addition, the transplantation efficiency in oriental bonito larvae into which primordial germ cells were transplanted at 7th day after fertilization was also higher than that of primordial germ cells transplanted at 3rd day after fertilization. The “transplantation efficiency” is the same as that described in Example 1. The above transplantation efficiency value is the average value of each transplantation efficiency value of three tests (n=3). These results showed that germ cell lineage can be efficiently obtained by transplanting isolated germ cells from testis into oriental bonito at a developmental stage after black pigmentary cells have developed in the retina and before multiple layers of germ cells are formed in the genitals.

### Example 3: Differentiation Induction of Primordial Germ Cells of Ninespine Stickleback

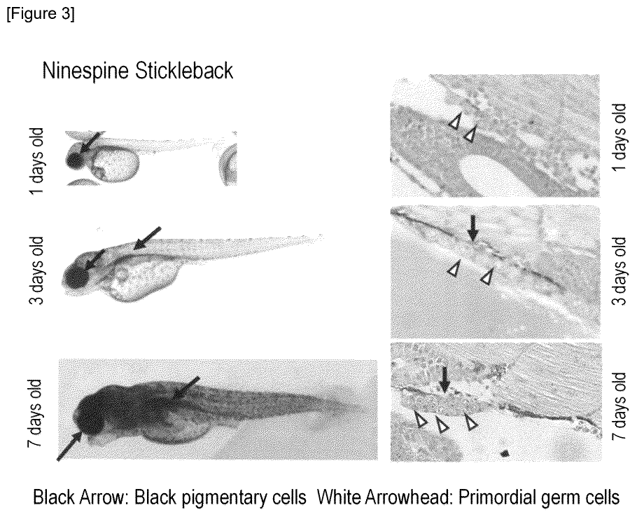

Following the same procedure as in Example 1, germ cells from testis of ninespine stickleback were isolated and transplanted into ninespine stickleback larvae at each growth stage at 1 day, 3 days and 7 days after fertilization (1 day old, 3 days old and 7 days old, respectively). In ninespine stickleback larvae at 1 day and 3 days after fertilization, black pigmentary cells were observed in the retina, and multiple layers of germ cells were not observed in the genital organs. On the other hand, in ninespine stickleback larvae at 7 days after fertilization, black pigmentary cells were observed in the retina, and multiple layers of germ cells were also observed in the genital organs. Photographs of ninespine stickleback at each growth stage are shown in FIG. 3.

It was confirmed that the transplanted germ cells from testis were induced to differentiate into germ cell lineage. In addition, in ninespine stickleback larvae into which primordial germ cells were transplanted at 1 day old and 3 day old, the transplantation efficiency of the differentiated germ cell lineage was approximately 15% and 20%, respectively. On the other hand, in ninespine stickleback larvae into which primordial germ cells were transplanted at 5 days old, the transplantation efficiency of the differentiated germ cell lineage was approximately 11%. The “transplantation efficiency” is the same as that described in Example 1. The above values of each transplantation efficiency are the average values of each transplantation efficiency of three tests (n=3). These results showed that germ cell lineage can be efficiently obtained by transplanting isolated germ cells into ninespine stickleback at a developmental stage after black pigmentary cells have developed in the retina and before multiple layers of germ cells are formed in the genitals.

### INDUSTRIAL APPLICABILITY

According to the present disclosure, the germ cell lineage of an oviparous vertebrate can be obtained more efficiently than with conventional techniques. There is a high demand for the germ cell lineage of an oviparous vertebrate for research purposes, for securing food resources, etc., and a method that can obtain the germ cell lineage of an oviparous vertebrate more efficiently than with conventional techniques is highly useful.

1-14. (canceled)

15. A method for inducing differentiation of isolated undifferentiated germ cells into germ cell lineage, comprising the step of:
(a) preparing a host fish at a developmental stage after the development of black pigmentary cells in the retina and before the formation of multiple layers of germ cells in the genitals; and
(b) transplanting isolated undifferentiated germ cells from a donor fish into the host fish,
the isolated undifferentiated germ cells comprise undifferentiated germ cells of the donor fish which are visualized and isolated,
wherein the visualization of the undifferentiated germ cells is performed
by incorporating a plasmid into which a gene encoding the fluorescent protein FP is incorporated into the regulatory region of the vasa gene into the cytoplasm of a fertilized ovum of the donor fish, or
by inoculating the donor fish with a labeled antibody that specifically recognizes an antigen on the surface of the cell membrane of the germ cells from the donor fish, and binding the labeled antibody to the antigen on the surface of the cell membrane of the germ cells from the donor fish.

16. The method according to claim 15, wherein the isolated undifferentiated germ cells comprise at least one type of cells selected from the group consisting of primordial germ cells, oogonia and spermatogonia.

17. The method according to claim 15, wherein the transplantation is carried out by transplanting the isolated undifferentiated germ cells into the abdominal cavity of the host fish.

18. The method according to claim 15, wherein the induction of differentiation of the isolated undifferentiated germ cells into the germ cell lineage is induction of differentiation of primordial germ cells into oocytes or spermatogonia, induction of differentiation of primordial germ cells into ovum or sperm, induction of differentiation of oocytes into ovum or sperm, or induction of differentiation of spermatogonia into ovum or sperm.

19. The method according to claim 15, wherein the donor fish and the host fish are of different strains or species.

20. The method according to claim 15, wherein the isolated undifferentiated germ cells are genetically modified.

21. The method according to claim 15, wherein the donor fish is in an embryonic state before or after hatching.

22. A method for producing a fish, comprising the steps of:
(a) inducing differentiation of isolated undifferentiated germ cells from a fish by the method according to claim 15 to obtain at least one of ovum and sperm; and
(b) obtaining an individual of a fish using at least one of the ovum and sperm obtained in the step (a).

23. A method for producing a genetically modified fish, comprising the steps of:
(a) inducing differentiation of isolated undifferentiated germ cells from a fish having a genetic modification by the method according to claim 15 to obtain at least one of ovum and sperm having a genetic modification; and
(b) obtaining an individual of a fish having the genetic modification using at least one of the ovum and sperm having the genetic modification obtained in the step (a).

24. The method according to claim 16, wherein the transplantation is carried out by transplanting the isolated undifferentiated germ cells into the abdominal cavity of the host fish.
